(19) United States
(12) Patent Application Publication
(10) Pub. No.: US 2015/0065867 A1
(43) Pub. Date: Mar. 5, 2015

(54) ULTRASOUND DIAGNOSTIC APPARATUS AND METHOD OF OPERATING THE SAME

(71) Applicant: SAMSUNG ELECTRONICS CO., LTD., Suwon-si (KR)

(72) Inventors: Jeong CHO, Seoul (KR); Eun-ho YANG, Seoul (KR); Ho-san HAN, Seoul (KR)

(73) Assignee: SAMSUNG ELECTRONICS CO., LTD., Suwon-si (KR)

(21) Appl. No.: 14/476,009

(22) Filed: Sep. 3, 2014

(30) Foreign Application Priority Data
Sep. 3, 2013 (KR) 10-2013-0105697

(51) Int. Cl. A61B 8/08; A61B 8/14; A61B 8/00

(52) U.S. Cl. CPC A61B 8/0841; A61B 8/462; A61B 8/464; A61B 8/14
USPC 600/424

(57) ABSTRACT

An ultrasound diagnostic apparatus and a method for operating the ultrasound diagnostic apparatus are provided, whereby a location of a needle or an insertion degree may be more conveniently and accurately determined when a user treats or diagnoses an object by inserting the needle into the object. The ultrasound diagnostic apparatus includes a first display device that displays a first ultrasound image and a second display device that operates as a battery, is wirelessly connected to a main body of the ultrasound diagnostic apparatus, and easily moves. The second display device displays an enlarged ultrasound image of a needle and is disposed near a patient.

### CROSS-REFERENCE TO RELATED APPLICATIONS

This application claims priority from Korean Patent Application No. 10-2013-0105697, filed on Sep. 3, 2013, in the Korean Intellectual Property Office, the disclosure of which is incorporated herein by reference in its entirety.

### BACKGROUND

1. Field

One or more exemplary embodiments relate to an ultrasound diagnostic apparatus and a method for operating the ultrasound diagnostic apparatus, and more particularly, to an ultrasound diagnostic apparatus which is usable for treatment or diagnosis to be carried out by inserting a needle into an object, and a method for operating the ultrasound diagnostic apparatus.

2. Description of the Related Art

Ultrasound systems irradiate an ultrasonic signal which is generated from a transducer of an ultrasound probe onto an internal part of an object and receive information which relates to an echo signal reflected from the internal part of the object, thereby acquiring an image of the internal part of the object. In particular, ultrasound systems are used for the medical purpose of observing the inside of an object, detecting a foreign material, and assessing an injury.

Ultrasound systems have stabilities which are typically higher than those of diagnostic apparatuses using X-rays, display an image in real time, and are safe because there is no exposure to radioactivity, and thus may be widely used in conjunction with other medical image diagnostic apparatuses.

When a user uses a syringe for treatment or a biopsy in a medical field, because an internal part of a patient, into which a needle of the syringe is inserted, is not seen, it may be difficult to determine a location of the inserted needle or an insertion degree. For example, when the user draws blood from a specific blood vessel of the patient, it may take a long time to determine an accurate location of the blood vessel or an insertion degree of the needle.

Therefore, the user may use an ultrasound system to carry out treatment or a biopsy that uses the syringe so as to reduce a time taken and increase accuracy. The ultrasound system provides images of the object and the needle inserted into the object, thereby increasing diagnosis or treatment accuracy.

However, a general ultrasound system displays an ultrasound image via a display unit 11 which is fixed to an ultrasound diagnostic apparatus 10, as shown in FIG. 1. Thus, for example, when the user uses an ultrasound probe 20 and a syringe 30 to carry out a biopsy of an object 105, because the user is provided with an ultrasound image via the display unit 11 which is disposed relatively far away from the user, it is difficult to accurately determine a location of a needle or an insertion degree.

Furthermore, it may be inconvenient for the user to acquire the ultrasound image of the object 105 by using the ultrasound probe 20 due to a communication cable which is used to connect the ultrasound probe 20 and the ultrasound diagnostic apparatus 10. In addition, there may be a problem in that it is unsanitary when the patient contacts the communication cable.

### SUMMARY

One or more exemplary embodiments include an ultrasound diagnostic apparatus and a method for operating the ultrasound diagnostic apparatus whereby a location of a needle or an insertion degree may be more conveniently and accurately determined when a user treats or diagnoses an object by inserting the needle into the object.

Additional aspects will be set forth in part in the description which follows and, in part, will be apparent from the description, or may be learned by practice of the presented exemplary embodiments.

According to one or more exemplary embodiments, there is provided an ultrasound diagnostic apparatus including: a transceiver configured to receive, from an ultrasound probe, ultrasound image data which relates to an object and to a needle which is inserted into the object; a first display device configured to display a first ultrasound image which is generated from the ultrasound image data; a needle detector configured to detect a region which corresponds to the needle from the first ultrasound image; a controller configured to generate a second ultrasound image which includes the region which corresponds to the needle based on an output from the needle detector; and a second display device configured to display the second ultrasound image.

The controller may be further configured to generate the second ultrasound image by enlarging a part of the first ultrasound image which includes the region which corresponds to the needle by a predetermined enlargement factor. The controller may be further configured to determine the enlargement factor based on a user input.

The needle detector may be further configured to detect a change in a location of an edge of the needle from the first ultrasound image, and the controller may be further configured to update the second ultrasound image based on the detected change in the location of the edge of the needle.

The second ultrasound image may include an emphasized portion which relates to the needle.

The needle detector may include a location detector configured to detect a location of the needle from within the object, and to detect the region which corresponds to the needle from the first ultrasound image based on the detected location of the needle.

The second display device may be further configured to wirelessly receive the second ultrasound image from the transceiver.

The second display device may be fixable to the ultrasound probe.

The transceiver may be further configured to wirelessly receive the ultrasound image data from the ultrasound probe.

According to one or more exemplary embodiments, there is provided a method for operating an ultrasound diagnostic apparatus, the method including: receiving, from an ultrasound probe, ultrasound image data which relates to an object and to a needle which is inserted into the object; displaying, on a first display device, a first ultrasound image which is generated from the received ultrasound image data; detecting a region which corresponds to the needle from the first ultrasound image; generating a second ultrasound image which includes the region which corresponds to the needle based on a result of the detecting; and displaying, on a second display device, the second ultrasound image.

The generating the second ultrasound image may include enlarging a part of the first ultrasound image which includes the region which corresponds to the needle by a predetermined enlargement factor.

The generating the second ultrasound image may include determining the enlargement factor based on a user input.

The detecting the region which corresponds to the needle may include detecting a change in a location of an edge of the needle from the first ultrasound image, and the generating the second ultrasound image may include updating the second ultrasound image based on the detected change in the location of the edge of the needle.

The generating the second ultrasound image may include including, within the second ultrasound image, an emphasized portion which relates to the needle.

The detecting the region which corresponds to the needle may include: detecting a location of the needle from within the object; and detecting the region which corresponds to the needle from the first ultrasound image based on the detected location of the needle.

The displaying the second ultrasound image may include wirelessly receiving the second ultrasound image by the second display device.

The second display device may be fixable to the ultrasound probe.

The receiving the ultrasound image data may include wirelessly receiving the ultrasound image data from the ultrasound probe.

According to one or more exemplary embodiments, there is provided a non-transitory computer-readable storage medium storing a computer program for executing the method of receiving, from an ultrasound probe, ultrasound image data relating to an object and to a needle inserted into the object; displaying, on a first display device, a first ultrasound image which is generated from the received ultrasound image data; detecting a region corresponding to the needle from the first ultrasound image; generating a second ultrasound image which includes the region which corresponds to the needle based on a result of the detecting; and displaying, on a second display device. the second ultrasound image.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a diagram of a general ultrasound system;

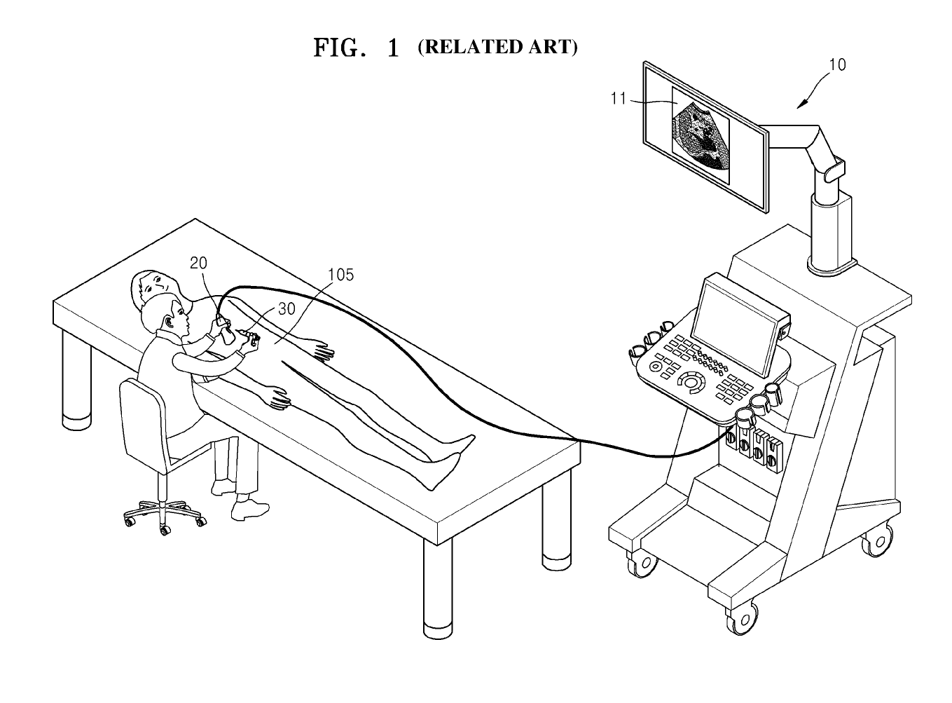

FIG. 2 is a diagram which illustrates an ultrasound system which includes an ultrasound probe and an ultrasound diagnostic apparatus, according to an exemplary embodiment;

FIG. 3 is a flowchart of a method for operating an ultrasound diagnostic apparatus, according to an exemplary embodiment;

FIGS. 4A and 4B are examples of screens displayed on a first display unit and a second display unit in connection with a method for operating an ultrasound diagnostic apparatus, according to an exemplary embodiment;

FIG. 5 is a block diagram of an ultrasound diagnostic apparatus, according to an exemplary embodiment; and

FIG. 6 is a block diagram of an ultrasound system which includes an ultrasound probe and an ultrasound diagnostic apparatus, according to an exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description.

Throughout the specification, when an element is referred to as being “connected” or “coupled” to another element, it may be “directly connected or coupled” to the other element or “electrically connected or coupled” with intervening elements.

When a part “includes” or “comprises” an element, unless there is a particular description contrary thereto, the part may further include other elements, not excluding the other elements.

Moreover, each of terms such as “ . . . unit” and “module” as described in specification denotes an element for performing at least one function or operation, and may be implemented in hardware, software or a combination of hardware and software.

The term “ultrasonic image” used herein denotes an image of an object which image is acquired by using an ultrasonic wave. Further, the term “object” as used herein may include an organic substance or an inorganic substance indicated by the image. The object may include a part of a physical body. For example, an object may include an organ such as a liver, a heart, a womb, a brain, breasts, an abdomen, or the like, or a fetus, and may include a cross-sectional surface of the physical body.

Moreover, the term “user” as used herein typically refers to a medical expert, and may be a doctor, a nurse, a medical technologist, a sonographer, a medical image expert, or the like. However, the user is not limited thereto.

The present inventive concept will now be described more fully with reference to the accompanying drawings, in which exemplary embodiments are shown. The present inventive concept may, however, be embodied in many different forms and should not be construed as being limited to the exemplary embodiments set forth herein; rather, these exemplary embodiments are provided so that this disclosure will be thorough and complete, and will fully convey the present inventive concept to those of ordinary skill in the art. In the following description, well-known functions or constructions are not described in detail since they would obscure the present disclosure with unnecessary detail. Throughout the specification, like reference numerals in the drawings denote like elements. As used herein, the term “and/or” includes any and all combinations of one or more of the associated listed items. Expressions such as “at least one of,” when preceding a list of elements, modify the entire list of elements and do not modify the individual elements of the list.

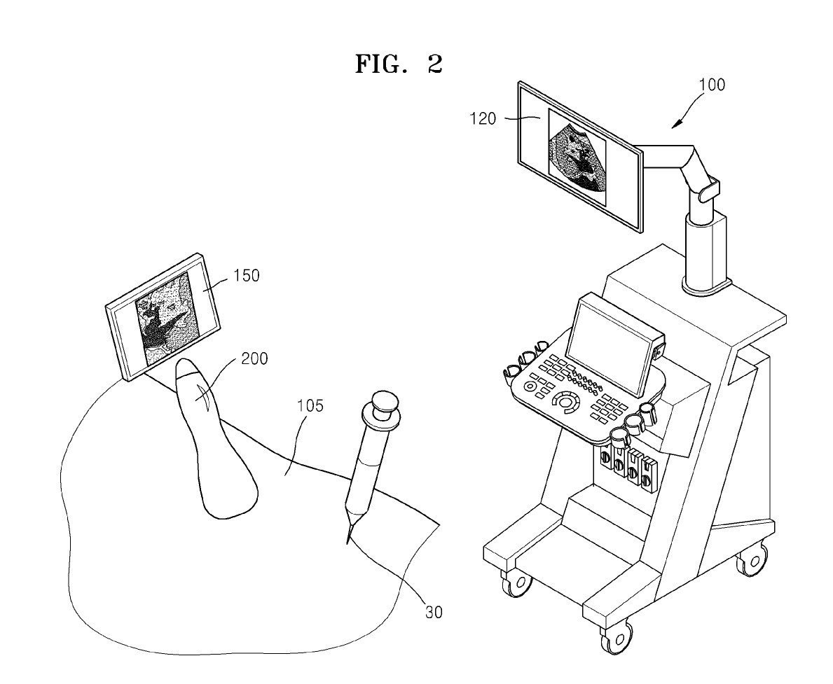

FIG. 2 is a diagram which illustrates an ultrasound system which includes an ultrasound probe 200 and an ultrasound diagnostic apparatus 100, according to an exemplary embodiment.

Referring to FIG. 2, the ultrasound system according to an exemplary embodiment includes the ultrasound diagnostic apparatus 100 and the ultrasound probe 200 that are connected to each other by wire or wirelessly.

The ultrasound probe 200 forms a receiving signal by transmitting an ultrasound signal to an object 105 based on a control signal which is received from the ultrasound diagnostic apparatus 100 and receiving the ultrasound signal (i.e. an ultrasound echo signal) which is reflected from the object 105. The ultrasound probe 200 focuses the receiving signal in order to form ultrasound image data, and then transmits the ultrasound image data to the ultrasound diagnostic apparatus 100.

The ultrasound diagnostic apparatus 100 forms an ultrasound image by using the ultrasound image data which is received from the ultrasound probe 200, and displays the ultrasound image on a first display unit (also referred to herein as a “first display device” and/or a “first display”) 120.

The ultrasound diagnostic apparatus 100 according to an exemplary embodiment provides a more convenient and accurate determination of a location of a needle 30 or an insertion degree when a user carries out treatment or diagnosis by inserting the needle 30 into the object 105.

To this end, the ultrasound diagnostic apparatus 100 may detect an image which indicates the needle 30 from within a first ultrasound image which is displayed on the first display unit 120. The ultrasound diagnostic apparatus 100 may select a partial region of the first ultrasound image which includes the image of the needle 30, and display a second ultrasound image which is obtained by enlarging the selected region of the first ultrasound image on a second display unit (also referred to herein as a “second display device” and/or a “second display”) 150.

Therefore, the user who uses the ultrasound diagnostic apparatus 100 according to an exemplary embodiment may be provided with an image which is obtained by enlarging the image of the needle 30 inserted into the object 105 via the second display unit 150, thereby increasing an accuracy of treatment or diagnosis using the needle 30.

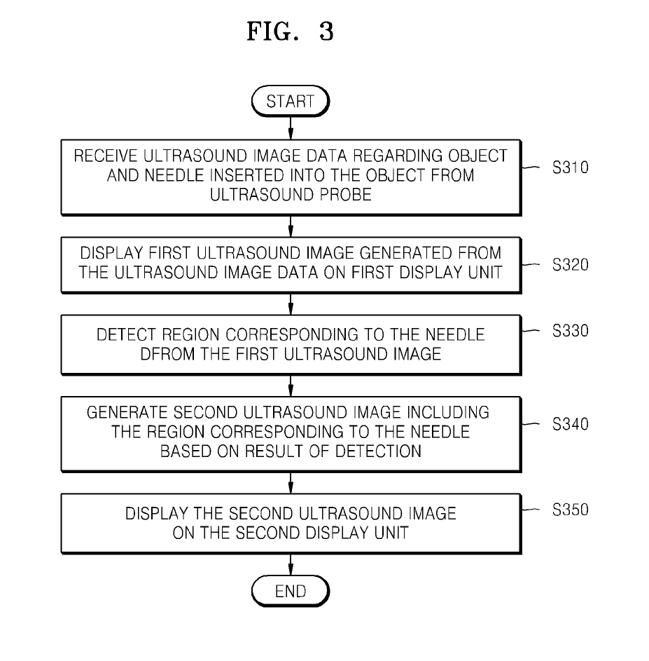

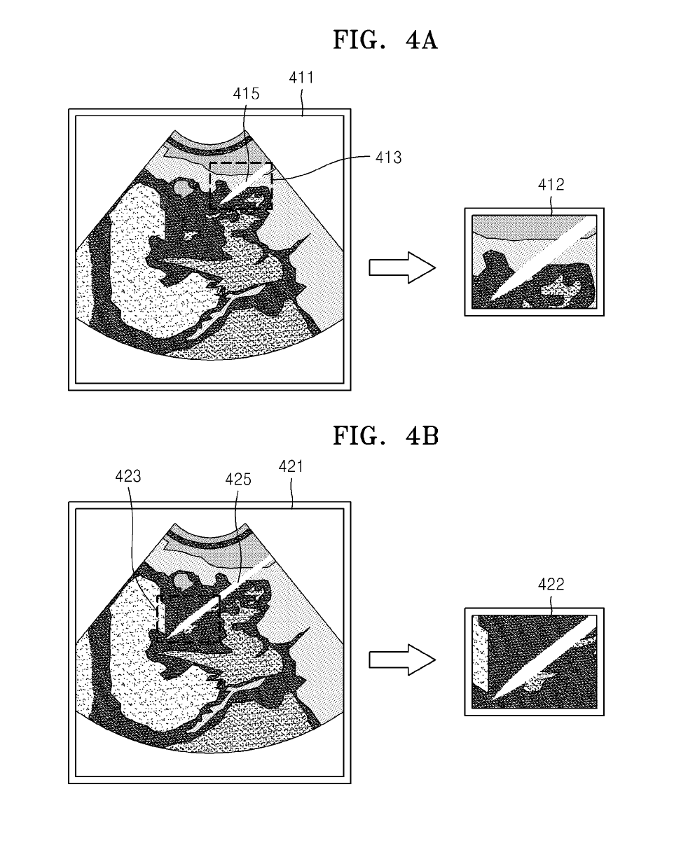

A method for operating the ultrasound diagnostic apparatus 100 according to an exemplary embodiment will now be described in detail with reference to FIGS. 3 and 4 below.

FIG. 3 is a flowchart of a method for operating the ultrasound diagnostic apparatus 100, according to an exemplary embodiment.

In operation S310, the ultrasound diagnostic apparatus 100 according to an exemplary embodiment receives, from the ultrasound probe 200, ultrasound image data which relates to an object and to a needle inserted into the object. A user may contact the ultrasound probe 200 on a surface of the object in a state where the needle is inserted into the object for treatment or diagnosis. The ultrasound probe 200 transceives an ultrasound signal to the object into which the needle is inserted and receives a resulting reflection ultrasound signal, thereby receiving the ultrasound image data which relates to the object and to the needle.

The ultrasound probe 200 that transmits the ultrasound image data to the ultrasound diagnostic apparatus 100 may be a wireless ultrasound probe. Thus, the ultrasound diagnostic apparatus 100 may wirelessly receive the ultrasound image data from the ultrasound probe 200.

In operation S320, the ultrasound diagnostic apparatus 100 according to an exemplary embodiment displays, on the first display unit 120, a first ultrasound image which is generated from the ultrasound image data. The ultrasound diagnostic apparatus 100 may generate the first ultrasound image from the ultrasound image data based on information which relates to the object and the needle which is included in the ultrasound image data. The ultrasound diagnostic apparatus 100 may generate the first ultrasound image by combining an object image which is generated from the ultrasound image data relating to the object and a needle image which is generated from the ultrasound image data relating to the needle.

In operation S330, the ultrasound diagnostic apparatus 100 according to an exemplary embodiment of the present invention detects a region which corresponds to the needle from the first ultrasound image. In this regard, the “region which corresponds to the needle” may refer to a partial region of the first ultrasound image and/or to a region which includes an image which is generated by using an ultrasound echo signal reflected from the needle. Alternatively, the “region which corresponds to the needle” may refer to a partial region of the first ultrasound image which includes a region which includes an image generated by using an ultrasound echo signal reflected from the needle or the object disposed near the needle.

For example, the ultrasound diagnostic apparatus 100 may detect the needle from the first ultrasound image and select a predetermined region of the first ultrasound image which includes at least a part of the detected needle. The ultrasound diagnostic apparatus 100 may determine the selected region of the first ultrasound image as the region corresponding to the needle.

As an example, the ultrasound diagnostic apparatus 100 may analyze the ultrasound image data and detect the region corresponding to the needle from the first ultrasound image. For example, the ultrasound diagnostic apparatus 100 may detect the region corresponding to the needle based on at least one from among brightness of the ultrasound image data, shapes of regions which indicate the same brightness, and a distribution of the regions which indicate the same brightness. In this aspect, the needle is generally displayed as being brighter than the object, and thus a structure which has a brightness level higher than a predetermined threshold brightness level in a straight line may be detected as the needle.

As another example, the ultrasound diagnostic apparatus 100 may detect the region corresponding to the needle based on information which relates to a location of the needle. The ultrasound diagnostic apparatus 100 may detect the location of the needle inserted into the object and detect the region corresponding to the needle from the first ultrasound image based on the detected location of the needle. In order to detect the location of the needle, the ultrasound probe 200 may include a needle location detection unit (also referred to herein as a “needle location detector”) (not shown).

For example, the ultrasound probe 200 may include a syringe that is combined with the ultrasound probe 200 and inserted into a body. The ultrasound probe 200 may detect movement information which relates to the syringe. In this regard, the movement information which relates to the syringe may be measured based on a variable resistance that is synchronized with a physical movement of the syringe and that has a variable resistance value. The needle location detection unit (not shown) included in the ultrasound probe 200 may calculate a depth and/or an angle of the needle inserted into the object based on the movement information which relates to the syringe, thereby detecting the location of the needle.

The ultrasound diagnostic apparatus 100 may detect the region which corresponds to the needle from the first ultrasound image and detect a point which corresponds to an edge of the needle from the detected region. The ultrasound diagnostic apparatus 100 may detect a predetermined region of the first ultrasound image which includes the detected edge of the needle as the region corresponding to the needle.

In operation S340, the ultrasound diagnostic apparatus 100 according to an exemplary embodiment generates a second ultrasound image which includes the region corresponding to the needle based on a result of the detection performed in operation S330.

The second ultrasound image may include an image which is obtained by enlarging a part of the first ultrasound image, which part includes the region corresponding to the needle, by a predetermined enlargement factor. The ultrasound diagnostic apparatus 100 may determine the enlargement factor based on a user input. The enlargement factor may be a previously stored value.

When the ultrasound diagnostic apparatus 100 wholly displays the first ultrasound image which is displayed on the first display unit 120 on the second display unit 150, the enlargement factor which relates to the first ultrasound image may be equal to 100%. Thus, when the ultrasound diagnostic apparatus 100 displays a partial image of the first ultrasound image which corresponds to one-fourth of the first ultrasound image on a whole screen of the second display unit 150 as the second ultrasound image, the enlargement factor which relates to the first ultrasound image may be equal to 400%. When the ultrasound diagnostic apparatus 100 displays a partial image of the first ultrasound image which corresponds to one-eighth of the first ultrasound image on a whole screen of the second display unit 150 as the second ultrasound image, the enlargement factor which relates to the first ultrasound image may be equal to 800%.

The ultrasound diagnostic apparatus 100 may generate a part of the first ultrasound image which part includes the region corresponding to the needle as the second ultrasound image. The ultrasound diagnostic apparatus 100 may detect a location change in the edge of the needle from the first ultrasound image and then update the second ultrasound image based on the detected location change in the edge of the needle.

For example, the ultrasound diagnostic apparatus 100 may select a partial region of the first ultrasound image that may be enlarged and displayed in such a manner that the edge of the needle is disposed at a predetermined location of the second ultrasound image. Thus, the edge of the needle which is inserted into the object changes, and thus the selected region of the first ultrasound image may be different. The selected region of the first ultrasound image is different, and thus the second ultrasound image displayed on the second display unit 150 is updated. An updating of the second ultrasound image will be described in more detail with reference to FIG. 4 below.

The ultrasound diagnostic apparatus 100 may generate the second ultrasound image such that an emphasized portion which relates to the needle is included in the image. In particular, the ultrasound diagnostic apparatus 100 may generate the second ultrasound image such that a color, a contrast, and/or a shape of an image which indicates the needle is corrected in such a manner that the needle and the object may be clearly distinguished from each other. For example, the ultrasound diagnostic apparatus 100 may correct the ultrasound image data and generate the second ultrasound image from the corrected ultrasound image data in such a manner that the image indicating the needle may be displayed to be brighter and distinguished from the image of the object which has a relatively low brightness.

In operation S350, the ultrasound diagnostic apparatus 100 according to an exemplary embodiment displays the second ultrasound image on the second display unit 150. In this regard, the second display unit 150 that displays the second ultrasound image may receive a control signal via the second display unit 150 or via a user input unit (also referred to herein as a “user input device”) (not shown) which is included in the ultrasound probe 200. For example, the ultrasound diagnostic apparatus 100 may receive the enlargement factor which relates to the first ultrasound image for generating the second ultrasound image via the second display unit 150 or via the user input unit (not shown) which is included in the ultrasound probe 200.

The second display unit 150 may wirelessly receive the second ultrasound image from a main body of the ultrasound diagnostic apparatus 100. The main body of the ultrasound diagnostic apparatus 100 includes the first display unit 120. The main body of the ultrasound diagnostic apparatus 100 performs functions of receiving the ultrasound image data from the ultrasound probe 200, displaying the first ultrasound image, and generating the second ultrasound image.

The ultrasound diagnostic apparatus 100 may process the ultrasound image data which is received from the ultrasound probe 200, and then wirelessly transmit a high resolution image to the second display unit 150. The main body of the ultrasound diagnostic apparatus 100 and the ultrasound probe 200 may be connected to each other in a wireless gigabit alliance (WiGig) manner in order to facilitate transmission and reception of the high resolution image.

In particular, the second display unit 150 may be configured as a wireless monitor that wirelessly receives the ultrasound image from the main body of the ultrasound diagnostic apparatus 100. The second display unit 150 may be wirelessly connected to the main body of the ultrasound diagnostic apparatus 100 so that the second display unit 150 may be placed at a location and an angle desired by the user. For example, the second display unit 150 may be located in a user's line of sight while the user is treating or diagnosing a patient by using the needle.

The second display unit 150 may be fixed to the ultrasound probe 200. Thus, the second display unit 150 may be small enough to be fixed to the ultrasound probe 200. A coupling unit (also referred to herein as a “coupler”) (not shown) that is mechanically coupled to the ultrasound probe 200 may be disposed on an outer surface of the second display unit 150. A coupling unit (also referred to herein as a “coupler”) (not shown) that is mechanically coupled to a tripod or an arm installed near the object as well as the ultrasound probe 200 may be disposed on the outer surface of the second display unit 150.

Therefore, the user who uses the ultrasound diagnostic apparatus 100 according to an exemplary embodiment may arrange the second display unit 150 that displays the second ultrasound image for guiding treatment or diagnosis using the needle to be located near the patient. Thus, the user may more easily and accurately determine a location of the needle and/or an insertion degree.

The second display unit 150 may display an enlarged ultrasound image that is automatically updated based on a change in the location of the needle. The second display unit 150 may further enlarge and display the second ultrasound image based on a user input. When the second display unit 150 enlarges and displays the second ultrasound image based on the user input, the second display unit 150 may enlarge the second ultrasound image with respect to a location of an edge of the needle. In particular, the second display unit 150 may enlarge and display the second ultrasound image in such a manner that the location of the edge of the needle of the second ultrasound image may be disposed on a predetermined location of a screen of the second display unit 150, for example, the edge of the needle may be centered with respect to the screen of the second display unit 150.

Therefore, the user who uses the ultrasound diagnostic apparatus 100 according to an exemplary embodiment may be provided with the second ultrasound image showing an enlarged and detailed location of the edge of the needle inside the object, thereby increasing an accuracy of treatment or diagnosis using the needle.

The second display unit 150 may operate by using a battery, and may be designed not to have any curves or joints. The second display unit 150 according to an exemplary embodiment may be easily sterilized, whereby the user may more sanitarily use the ultrasound diagnostic apparatus 100.

FIGS. 4A and 4B are examples of screens which are respectively displayed on the first display unit 120 and the second display unit 150 in connection with a method for operating the ultrasound diagnostic apparatus 100, according to an exemplary embodiment.

Referring to FIG. 4A, the ultrasound diagnostic apparatus 100 may detect a region 415 which corresponds to a needle inserted into an object from a first ultrasound image 411 which is displayed on the first display unit 120. The ultrasound diagnostic apparatus 100 may select a partial region 413 of the first ultrasound image 411 for generating a second ultrasound image 412 based on the detected region 415 which corresponds to the needle and a previously set enlargement factor. The ultrasound diagnostic apparatus 100 may generate the second ultrasound image 412 which includes the partial region 413 of the first ultrasound image 411. The ultrasound diagnostic apparatus 100 may display the second ultrasound image 412 on the second display unit 150.

Referring to FIG. 4B, the ultrasound diagnostic apparatus 100 may detect a region 425 which corresponds to the needle inserted into the object from a first ultrasound image 421 which is displayed on the first display unit 120. The ultrasound diagnostic apparatus 100 may change a partial region 423 of the first ultrasound image 421 which partial region 423 is selected in order to generate a second ultrasound image 422 based on a change in a location of an edge of the needle. Thus, when the location of an edge of the needle is changed because the needle is deeply inserted into the object, the ultrasound diagnostic apparatus 100 may automatically update the second ultrasound image 422 which is displayed on the second display unit 150. The second display unit 150 may display the updated second ultrasound image 422.

Therefore, the ultrasound diagnostic apparatus 100 according to an exemplary embodiment provides a second ultrasound image that is automatically updated based on the change in the edge of the needle via the second display unit 150, thereby providing a user with an environment in which the user may accurately treat or diagnose a patient by using the needle.

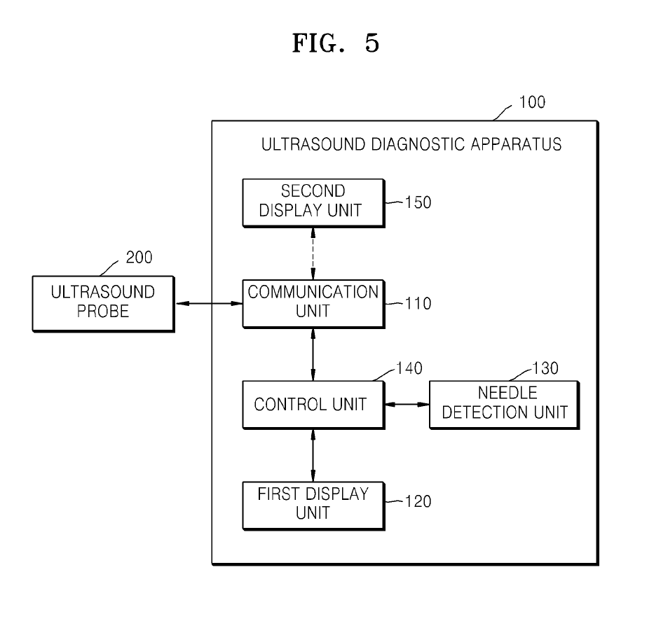

FIG. 5 is a block diagram of an ultrasound diagnostic apparatus 100, according to an exemplary embodiment.

Elements of the ultrasound diagnostic apparatus 100 according to an exemplary embodiment are configured to perform the operations of the method for operating the ultrasound diagnostic apparatus 100 which is illustrated in FIG. 3. Thus, although omitted below, the descriptions of the method for operating the ultrasound diagnostic apparatus 100 of FIG. 3 above may apply to the ultrasound diagnostic apparatus 100 of FIG. 5.

Referring to FIG. 5, the ultrasound diagnostic apparatus 100 according to an exemplary embodiment includes a communication unit (also referred to herein as a “communicator” and/or as a “transceiver”) 110, the first display unit (also referred to herein as “the first display device” and/or as “the first display”) 120, a needle detection unit (also referred to herein as a “needle detector”) 130, a control unit (also referred to herein as a “controller”) 140, and the second display unit (also referred to herein as “the second display device” and/or as “the second display”) 150.

The communication unit 110 receives ultrasound image data which relates to an object and ultrasound image data which relates to a needle inserted into the object from the ultrasound probe 200.

The first display unit 120 displays the first ultrasound image which is generated from the ultrasound image data.

The needle detection unit 130 detects a region which corresponds to the needle from the first ultrasound image. The needle detection unit 130 may include a location detection unit (also referred to herein as a “location detector”) (not shown) that detects a location of the needle with respect to the object. The needle detection unit 130 may detect the region corresponding to the needle from the first ultrasound image based on the detected location of the needle. The needle detection unit 130 may analyze the ultrasound image data and detect an ultrasound image which corresponds to the needle.

The control unit 140 generates a second ultrasound image by enlarging at least a part of the first ultrasound image based on a result of detection of the needle detection unit 130. The control unit 140 may generate a part of the first ultrasound image which part includes the region corresponding to the needle as the second ultrasound image. The control unit 140 may determine an enlargement factor which is used to enlarge the at least part of the first ultrasound image on the second ultrasound image based on a user input. The control unit 140 may update the second ultrasound image based on a change in the edge of the needle on the first ultrasound image. The control unit 140 may generate the second ultrasound image such that an emphasized portion which relates to the region corresponding to the needle is included in the second ultrasound image.

The second display unit 150 displays the second ultrasound image. In this regard, the second display unit 150 may wirelessly receive the second ultrasound image that is based on the ultrasound data transmitted from the communication unit 110 and is generated by the control unit 140. The second display unit 150 may include a WiGig communication module in order to receive the second ultrasound image that has a high resolution.

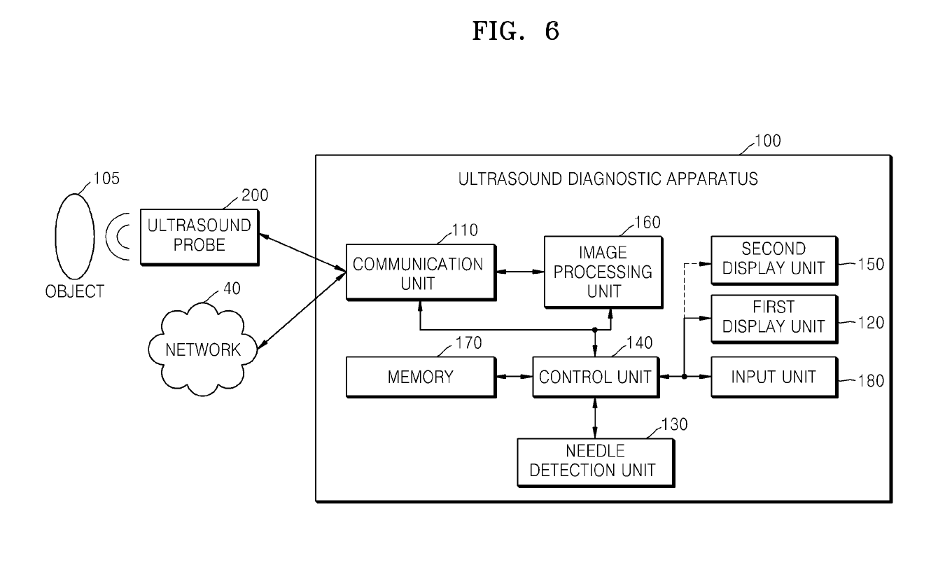

FIG. 6 is a block diagram of an ultrasound system which includes the ultrasound probe 200 and the ultrasound diagnostic apparatus 100, according to an exemplary embodiment.

Referring to FIG. 6, the ultrasound system may include the ultrasound probe 200 and the ultrasound diagnostic apparatus 100, according to an exemplary embodiment.

The ultrasound diagnostic apparatus 100 according to an exemplary embodiment may include an image processing unit (also referred to herein as an “image processor”) 160, a memory 170, and an input unit (also referred to herein as an “input device”) 180 in addition to the communication unit 110, the first display unit 120, the needle detection unit 130, the control unit 140, and the second display unit 150.

The ultrasound diagnostic apparatus 100 may be configured as a cart type diagnostic apparatus and/or as a portable diagnostic apparatus. The portable diagnostic apparatus may include any one or more of a picture archiving and communication system (PACS) viewer, a hand-carried cardiac ultrasound (HCU) device, a smart phone, a lap-top computer, a personal digital assistant (PDA), and a tablet personal computer (PC), but is not limited thereto.

The communication unit 110 may perform wired and/or wireless communication with the ultrasound probe 200. The communication unit 110 may transmit a control signal which is received from the control unit 140 to the ultrasound probe 200, and receive ultrasound image data which is transmitted by the ultrasound probe 200.

The communication unit 110 may be connected to a network 40 by wire or wirelessly and communicate with an external device and/or a server. The communication unit 110 may transmit and receive data to and from a hospital server or other medical devices of a hospital via a PACS. The communication unit 110 may perform data communication based on the digital imaging and communications in medicine (DICOM) standard.

The communication unit 110 may transmit and receive data which is associated with a diagnosis of the object, such as an ultrasound image of the object, ultrasound data, and Doppler data, over the network 40, and may also transmit and receive a medical image which is captured by another medical apparatus such as a computed tomography (CT) apparatus, a magnetic resonance imaging (MRI) apparatus, an X-ray apparatus, and/or the like. Furthermore, the communication unit 110 may receive a diagnosis history and/or a treatment schedule of a patient from the server, and use the received diagnosis history and/or treatment schedule in diagnosing the object. The communication unit 110 may communicate with a mobile terminal of a doctor or a customer, in addition to the server and/or the medical apparatus of the hospital.

The communication unit 110 may use short distance communication, such as wireless LAN, Wi-Fi, Bluetooth, Zigbee, Wi-Fi direct (WFD), ultra wideband (UWB), infrared data association (IrDA), Bluetooth low energy (BLE), near field communication (NFC), and/or the like, but the communication unit 110 is not limited thereto.

Wired communication technology used by the communication unit 110 may include any one or more of a pair cable, a coaxial cable, an optical fiber cable, an Ethernet cable, and the like.

Mobile communication technology used by the communication unit 110 may be employed for transmitting and/or receiving a wireless signal with at least one of a base station of a mobile communication network, an external terminal, and a server. In this regard, the wireless signal may include any one or more of a voice signal, a video call signal, and/or various forms of data according to transmission and receiving of text and multimedia messages.

The control unit 140 may generally control operations of the ultrasound diagnostic apparatus 100. In particular, the control unit 140 may control operations between the ultrasound probe 200, the communication unit 110, the first display unit 120, the needle detection unit 130, the control unit 140, the second display unit 150, the image processing unit 160, the memory 170, and the input unit 180.

The image processing unit 160 may generate and display an ultrasound image by scanning and converting the transmission data received from the ultrasound probe 200 via the communication unit 110. The ultrasound image may include a gray scale ultrasound image which is obtained by scanning the object according to an amplitude A mode, a brightness B mode, and a motion M mode as well as a Doppler image of a motion of the object. The Doppler image may include a blood flow Doppler image (also referred to as a color Doppler image) which shows a blood flow, a tissue Doppler image which shows a motion of a tissue, and a spectral Doppler image which shows a wave type moving speed of the object.

The memory 170 stores any one or more of various types of information which are processed by the ultrasound diagnostic apparatus 100. For example, the memory 170 may store medical data which is associated with a diagnosis of the object, such as input and output ultrasound data, and an ultrasound image, and may also store an algorithm or a program which is executed in the ultrasound diagnostic apparatus 100.

The memory 170 may be configured as any one or more of various types of storage media such as flash memory, hard disk, EEPROM, and/or any other suitable type of storage medium. The ultrasound diagnostic apparatus 100 may operate web storage and/or a cloud server that performs a storage function of the memory 170 on the web.

The first display unit 120 and the second display unit 150 may display the generated ultrasound image. The first display unit 120 and the second display unit 150 may display any one or more of various types of information which are processed by the ultrasound diagnostic apparatus 100 on a screen via a user interface (UI) or a graphic user interface (GUI), in addition to displaying the ultrasound image. The ultrasound diagnostic apparatus 100 may include an additional display unit based on a configuration shape.

The input unit 180 is used to receive, from a user, an input of data which relates to controlling the ultrasound diagnostic apparatus 100. The input unit 180 includes a unit configured for receiving an enlargement factor which relates to an enlargement of a second ultrasound image with respect to a first ultrasound image. The input unit 180 may include a hardware element such as any one or more of a key pad, a mouse, a touch panel, a touch screen, a trackball, and a jog switch, but the input unit 180 is not limited thereto. The input unit 180 may further include any one or more of various types of input units, such as an ECG measurement module, a respiration measurement module, a voice recognition sensor, a gesture recognition sensor, a finger print recognition sensor, an iris recognition sensor, a depth sensor, a distance sensor, and the like.

The ultrasound probe 200, the communication unit 110, the first display unit 120, the needle detection unit 130, the second display unit 150, the image processing unit 160, the memory 170, and the input unit 180 may wholly or partly operate by using a software module, but these elements are not limited thereto, and may partly operate by using hardware. At least one of the communication unit 110, the needle detection unit 130, the image processing unit 160, and the memory 170 may be included in the control unit 140, but the ultrasound diagnostic apparatus 100 is not limited thereto. At least one of the communication unit 110, the control unit 140, the image processing unit 160, and the memory 170 may be included in the ultrasound probe 200, but exemplary embodiments are not limited thereto.

The ultrasound probe 200 sends an ultrasound signal to the object 105 based on a control signal which is received from the ultrasound diagnostic apparatus 100, and receives an echo signal which is reflected from the object 105. The ultrasound probe 200 includes a plurality of transducers. The transducers may vibrate based on transferred electrical signals, and generate ultrasound waves that include acoustic energy.

The ultrasound probe 200 generates pulses which are used to form transmission ultrasound waves based on a predetermined pulse repetition frequency (PRF) in accordance with the control signal which is received from the ultrasound diagnostic apparatus 100. The ultrasound probe 200 applies a delay time that is used to determine a transmission directionality to the pulses. Each pulse to which the delay time is applied corresponds to each of a plurality of piezoelectric vibrators which are included in the transducers. The ultrasound probe 200 applies the pulses which correspond to the piezoelectric vibrators at a time which corresponds to each pulse to which the delay time is applied.

The ultrasound probe 200 may generate the transmission data by processing the echo signal reflected from the object 105. The ultrasound probe 200 may amplify the echo signal for each channel and perform an analog-digital conversion on the amplified echo signal. The ultrasound probe 200 may apply the delay time that is used to determine the transmission directionality to the digitally converted echo signal, and generate the transmission data by summing the echo signal to which the delay time is applied.

As described above, the ultrasound diagnostic apparatus 100 according to an exemplary embodiment includes the second display unit 150 that operates as a battery, is wirelessly connected to a main body of the ultrasound diagnostic apparatus 100, and easily moves, thereby facilitating a biopsy or treatment which is performable by using the ultrasound system.

The ultrasound diagnostic apparatus 100 according to an exemplary embodiment may include the ultrasound probe 200 being disposed near a patient and the second display unit 150 being easily fixable to a tripod or an arm. The second display unit 150 may display a first ultrasound image which is displayed on the first display unit 120 and may also display a second ultrasound image which is obtained by enlarging a part of the first ultrasound image by an enlargement factor desired by the user.

When the second display unit 105 displays the second ultrasound image obtained by enlarging the designated part of the first ultrasound image, the second display unit 105 may automatically change the part of the first ultrasound image that is designated with respect to the second ultrasound image based on a change in a location of an edge of a needle as shown on the first ultrasound image.

Therefore, the ultrasound diagnostic apparatus 100 according to an exemplary embodiment includes the second display unit 150 that displays an enlarged ultrasound image of the needle and is disposed near the patient, thereby facilitating the biopsy or the treatment which is performable by using the ultrasound system and/or increasing an accuracy thereof.

In addition, other exemplary embodiments can also be implemented through computer readable code/instructions in/on a medium, e.g., a transitory or non-transitory computer readable medium, in order to control at least one processing element to implement any of the above-described exemplary embodiments. The medium can correspond to any medium/media which permits the storage and/or transmission of the computer readable code.

The computer readable code can be recorded/transferred on a medium in any one or more of a variety of ways, with examples of the medium including recording media, such as magnetic storage media (e.g., read-only memory (ROM), floppy disks, hard disks, etc.) and optical recording media (e.g., compact disk-read-only memory (CD-ROMs), or digital versatile disks (DVDs)), and transmission media such as Internet transmission media. Thus, the medium may be such a defined and measurable structure which includes or carries a signal or information, such as a device carrying a bitstream, according to one or more exemplary embodiments. The medium may also be a distributed network, so that the computer readable code is stored/transferred and executed in a distributed fashion. Furthermore, the processing element could include a processor and/or a computer processor, and processing elements may be distributed and/or included in a single device.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope of the present inventive concept as defined by the following claims.

What is claimed is:

1. An ultrasound diagnostic apparatus comprising:
a transceiver configured to receive, from an ultrasound probe, ultrasound image data which relates to an object and to a needle which is inserted into the object;
a first display device configured to display a first ultrasound image which is generated from the ultrasound image data;
a needle detector configured to detect a region which corresponds to the needle from the first ultrasound image;
a controller configured to generate a second ultrasound image which includes the region which corresponds to the needle based on an output from the needle detector; and
a second display device configured to display the second ultrasound image.

2. The ultrasound diagnostic apparatus of claim 1, wherein the controller is further configured to generate the second ultrasound image by enlarging a part of the first ultrasound image which includes the region which corresponds to the needle by a predetermined enlargement factor.

3. The ultrasound diagnostic apparatus of claim 2, wherein the controller is further configured to determine the enlargement factor based on a user input.

4. The ultrasound diagnostic apparatus of claim 1, wherein the needle detector is further configured to detect a change in a location of an edge of the needle from the first ultrasound image, and
wherein the controller is further configured to update the second ultrasound image based on the detected change in the location of the edge of the needle.

5. The ultrasound diagnostic apparatus of claim 1, wherein the second ultrasound image includes an emphasized portion which relates to the needle.

6. The ultrasound diagnostic apparatus of claim 1, wherein the needle detector comprises a location detector configured to detect a location of the needle from within the object, and to detect the region which corresponds to the needle from the first ultrasound image based on the detected location of the needle.

7. The ultrasound diagnostic apparatus of claim 1, wherein the second display device is further configured to wirelessly receive the second ultrasound image from the transceiver.

8. The ultrasound diagnostic apparatus of claim 1, wherein the second display device is fixable to the ultrasound probe.

9. The ultrasound diagnostic apparatus of claim 1, wherein the transceiver is further configured to wirelessly receive the ultrasound image data from the ultrasound probe.

10. A method for operating an ultrasound diagnostic apparatus, the method comprising:
receiving, from an ultrasound probe, ultrasound image data which relates to an object and to a needle which is inserted into the object;
displaying, on a first display device, a first ultrasound image which is generated from the received ultrasound image data;
detecting a region which corresponds to the needle from the first ultrasound image;
generating a second ultrasound image which includes the region which corresponds to the needle based on a result of the detecting; and
displaying, on a second display device, the second ultrasound image.

11. The method of claim 10, wherein the generating the second ultrasound image comprises enlarging a part of the first ultrasound image which includes the region which corresponds to the needle by a predetermined enlargement factor.

12. The method of claim 11, wherein the generating the second ultrasound image comprises determining the enlargement factor based on a user input.

13. The method of claim 10, wherein the detecting the region which corresponds to the needle comprises detecting a change in a location of an edge of the needle from the first ultrasound image, and
wherein the generating the second ultrasound image comprises updating the second ultrasound image based on the detected change in the location of the edge of the needle.

14. The method of claim 10, wherein the generating the second ultrasound image comprises including, within the second ultrasound image, an emphasized portion which relates to the needle.

15. The method of claim 10, wherein the detecting the region which corresponds to the needle comprises:
detecting a location of the needle from within the object; and
detecting the region which corresponds to the needle from the first ultrasound image based on the detected location of the needle.

16. The method of claim 10, wherein the displaying the second ultrasound image comprises wirelessly receiving the second ultrasound image by the second display device.

17. The method of claim 10, wherein the second display device is fixable to the ultrasound probe.

18. The method of claim 10, wherein the receiving the ultrasound image data comprises wirelessly receiving the ultrasound image data from the ultrasound probe.

19. A non-transitory computer-readable storage medium storing a computer program for executing a method of operating an ultrasound diagnostic apparatus, the method comprising:
receiving, from an ultrasound probe, ultrasound image data which relates to an object and to a needle inserted into the object;
displaying, on a first display device, a first ultrasound image which is generated from the ultrasound image data;
detecting a region which corresponds to the needle from the first ultrasound image;
generating a second ultrasound image which includes the region which corresponds to the needle based on a result of the detecting; and
displaying, on a second display device, the second ultrasound image.

20. An ultrasound diagnostic apparatus comprising:
a transceiver configured to receive, from an ultrasound probe, ultrasound image data which relates to an object and to a needle which is inserted into the object;
a first display device configured to display a first ultrasound image which is generated from the ultrasound image data;
an image processor configured to generate a second ultrasound image which includes a region which corresponds to a location of the needle, which location is determined based on the first ultrasound image; and
a second display device configured to display the second ultrasound image.

21. The ultrasound diagnostic apparatus of claim 20, wherein the image processor is further configured to use a needle detector to determine the location of the needle and to select the region based on the determined location.

22. The ultrasound diagnostic apparatus of claim 21, wherein the image processor is further configured to emphasize the determined location of the needle within the second ultrasound image by using at least one from among a color which indicates the needle, a contrast which indicates the needle, and a shape which indicates the needle.

23. The ultrasound diagnostic apparatus of claim 20, wherein the image processor is further configured to generate the second ultrasound image by enlarging a part of the first ultrasound image which corresponds to the region by a predetermined enlargement factor.
